(19) United States
(12) Patent Application Publication
(10) Pub. No.: US 2022/0218861 A1
(43) Pub. Date: Jul. 14, 2022

(54) AIR PURIFIER COMBINED WITH AROMATIZING DEVICE

(71) Applicants: HSUAN-YU CHAO, TAIPEI (TW); MEI-MEI TSAI, TAIPEI (TW)

(72) Inventor: HSUAN-YU CHAO, TAIPEI (TW)

(21) Appl. No.: 17/148,547

(22) Filed: Jan. 13, 2021

(51) Int. Cl. A61L 9/12; B01D 46/00; B01D 46/12

(52) U.S. Cl. CPC A61L 9/122; B01D 46/0028; B01D 46/12; B01D 2267/60; A61L 2209/14; B01D 2265/06; B01D 46/0024

(57) ABSTRACT

Provided is an air purifier combined with an aromatizing device, including at least one air purifying unit having a lower housing, an upper housing, a fan, a filter, a filter cover, an aromatizing device, and a bottom cover. The lower housing is a hollow housing having a lower accommodating space for receiving the filter and the aromatizing device and is provided with air inlets at circumferences thereof for allowing external air to enter the air purifying unit. The filter is a hollow and cylindrical filter body having a hollow space for filtering external air. The aromatizing device is disposed within the hollow space of the filter for aromatizing purified air from the filter to generate purified and aromatized air. The upper housing is a hollow housing having an upper accommodating space for receiving the fan and is provided with air outlets at circumferences thereof for discharging purified and aromatized air.

### FIELD OF THE INVENTION

The invention is related to an air purifier, and more particularly to an air purifier combined with an aromatizing device.

### BACKGROUND OF THE INVENTION

Nowadays, the air quality in most of the countries around the world is hardly satisfactory. In particular, the problem of air pollution is quite severe in developing countries. Hundreds of pollutants and toxic substances floating in the air are impairing the human health. Especially, the COVID-19 pandemic has broken forth since the end of 2019. This novel coronavirus is found to invade the human body through the respiratory system. Thus far the COVID-19 pandemic situation has been deteriorating ever since the outbreak of the pandemic. The world would have to face other unknown contagious diseases that would jeopardize the whole human race. Hence, air purifiers are essential for the sterilization and disinfection of air.

The deterioration of outdoor air quality is partly owing to the combustion of fossil fuel (such as coal) used for generating electricity and exhaust discharged from factories, foundries, and vehicles. These would generate a large quantity of carbon dioxide (CO2), carbon oxide (CO), nitrogen oxide (NOx), particulate matter 2.5, dust, smog, and total volatile organic compound (TVOC) that can negatively affect the human health.

Moreover, the household furniture can incessantly emit various kinds of toxic compounds, and the toxic air pollutants from the outdoor environment can enter the house, office, hotel, and shopping mall through windows and ventilation system. As a result, the toxic substance can be mixed with bacteria, funguses, or organic compounds, which would in turn cause the concentration of the toxic air pollutant in the indoor air to be higher than the concentration of the toxic air pollutant in the outdoor air. Thus, the air purifier is significantly important to the daily life.

The air purifier has become a must-have appliance for families. The air purifier can remove dust mites and toxic substances, and help people alleviate allergic reactions and lower the possibility of having respiratory disease. If an aromatizing device is combined with an air purifier, people can breathe purified and aromatized air in the indoor environment. This can comfort people and effectively elevate the air quality by sterilizing and aromatizing the air.

Hence, it is necessary to develop an air purifier combined with an aromatizing device. The invention can meet this need.

### SUMMARY OF THE INVENTION

In view thereof, an object of the invention is to provide an air purifier combined with an aromatizing device for aromatizing air while purifying air, thereby discharging purified and aromatized air.

In an exemplary embodiment of the invention, an air purifier combined with an aromatizing device is provided, which includes: at least one air purifying unit having a lower housing, an upper housing, a fan, a filter, a filter cover, an aromatizing device, and a bottom cover, wherein:

the lower housing is a hollow housing having a lower accommodating space provided with a lower opening, such that the lower accommodating space is configured to receive the filter and the aromatizing device. The bottom surface of the lower housing is provided with air inlets that are spaced apart from each other on one side thereof for allowing the external air to enter the air purifying unit;

the upper housing is a hollow housing having an upper accommodating space and provided with a switch located on a top surface thereof. The switch is used to turn on or off the air purifying unit. The top surface of the upper housing is provided with air outlets that are spaced apart from each other on one side thereof for discharging purified and aromatized air;

the fan is mounted within the upper accommodating space of the upper housing for blowing the purified and aromatized air to the air outlets;

the filter is a hollow and cylindrical filter body having a hollow space for filtering the external air;

the filter cover is an annular body disposed between the lower side of the filter and the lower opening of the lower housing;

the aromatizing device is composed of an aromatizing piece and a mounting seat, both of which are disposed within the hollow space of the filter for aromatizing the purified air from the filter to generate purified and aromatized air;

the bottom cover is used to seal the hollow portion of the filter cover.

The air purifying unit further includes a first extension connector mounted on the top surface of the upper housing and a second extension connector mounted on the bottom surface of the lower housing. The first extension connector and the second extension connector are mated in a male-to-female configuration, such that the air purifying unit can be used solely, or jointly by vertically cascading a multiplicity of the air purifying units.

In a preferred embodiment of the invention, the upper housing includes a control circuit board mounted therein and connected to the switch, the first extension connector, the second extension connector, and the fan. The control circuit board is mounted on the bottom surface of the switch, and the fan is mounted below the control circuit board and is close to the air outlets.

In a preferred embodiment of the invention, the control circuit board can select the aromatizing mode of the air purifier through an application (app) of a mobile device by network transmission signals.

In a preferred embodiment of the invention, the filter is made up of a filter material selected from the group consisting of a high efficiency particulate air (HEPA) filter, an ultra low penetration air (ULPA) filter, nano photocatalysts, an active carbon filter, and an electrostatic filter.

In a preferred embodiment of the invention, the top surface of the mounting seat is provided with a round groove for allowing the aromatizing piece to be mounted therein. The bottom surface of the mounting seat is provided with a multiplicity of legs extending downwardly. The top surface of the bottom cover is provided with a multiplicity of hollow cylinders extending upwardly for receiving the legs of the mounting seat to secure the mounting seat.

In a preferred embodiment of the invention, a lucid ring body is mounted between the upper housing and the lower housing. Also, a light-emitting diode (LED) is electrically connected to the control circuit board and is disposed on the inside of the lucid ring body.

Next, a preferred embodiment will be given below with reference to the accompanying drawings to describe the invention in detail.

### BRIEF DESCRIPTION OF THE DRAWINGS

FIG. 1 shows an exploded view of the air purifier combined with an aromatizing device according to the invention.

FIG. 2 shows a cross-sectional view of the air purifier combined with an aromatizing device according to the invention, in which the switch of the air purifier is not depressed.

FIG. 3 shows cross-sectional view of the air purifier combined with an aromatizing device according to the invention, in which the switch of the air purifier is depressed such that the air purifier is working.

FIG. 4 is a schematic diagram showing the air purifier combined with an aromatizing device being controllable by a mobile device.

FIG. 5 is a schematic diagram showing the air purifier being implemented by cascading a multiplicity of air purifying units.

### DETAILED DESCRIPTION OF PREFERRED EMBODIMENTS

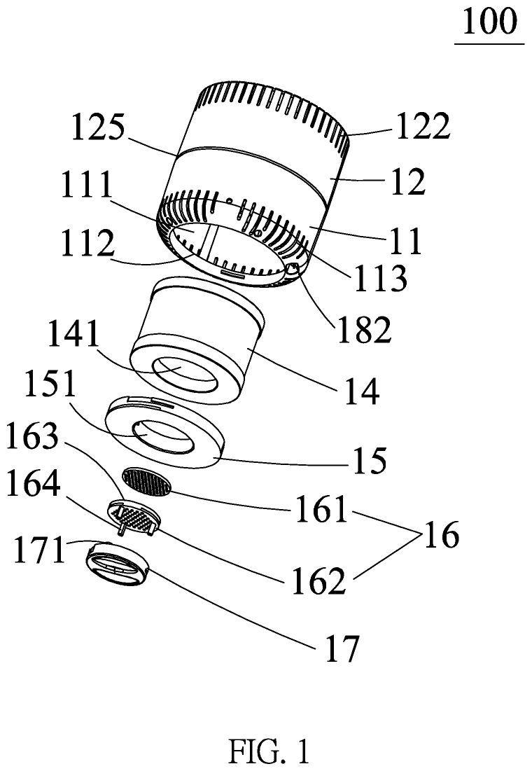

A preferred embodiment of the invention will be given below. Please refer to FIGS. 1-3, in which FIG. 1 shows an exploded view of the air purifier according to the invention. As shown, the inventive air purifier includes at least one air purifying unit 100 having a lower housing 11, an upper housing 12, a fan 13, a filter 14, a filter cover 15, an aromatizing device 16, and a bottom cover 17.

The lower housing 11 is a hollow housing having a lower accommodating space 111 provided with a lower opening 112, such that the lower accommodating space 111 is configured to receive the filter 14 and the aromatizing device 16. The bottom surface of the lower housing 11 is provided with air inlets 113 that are spaced apart from each other on one side thereof for allowing the external air to enter the air purifying unit 100.

The upper housing 12 is a hollow housing having an upper accommodating space 121 and provided with a switch 123 located on a top surface thereof. The switch 123 is used to turn on or off the air purifying unit 100. The top surface of the upper housing 12 is provided with air outlets 122 that are spaced apart from each other on one side thereof for discharging purified and aromatized air.

The fan 13 is mounted within the upper accommodating space 121 of the upper housing 12 for blowing the purified and aromatized air to the air outlets 122.

The filter 14 is a hollow and cylindrical filter body having a hollow space 141 for filtering the external air.

The filter cover 15 is an annular body disposed between the lower side of the filter 14 and the lower opening 112 of the lower housing 11.

The aromatizing device 16 is composed of an aromatizing piece 161 and a mounting seat 162, both of which are disposed within the hollow space 141 of the filter 14 for aromatizing the purified air from the filter 14 to generate purified and aromatized air.

The bottom cover 17 is used to seal the hollow portion 151 of the filter cover 15.

The air purifying unit 100 further includes a first extension connector 181 mounted on the top surface of the upper housing 12 and a second extension connector 182 mounted on the bottom surface of the lower housing 11. The first extension connector 181 and the second extension connector 182 are mated in a male-to-female configuration, such that the air purifying unit 100 can be used solely, or jointly by vertically cascading a multiplicity of the air purifying units 100.

In a preferred embodiment of the invention, the upper housing 12 includes a control circuit board 19 mounted therein and connected to the switch 123, the first extension connector 181, the second extension connector 182, and the fan 13. The control circuit board 19 is mounted on the bottom surface of the switch 123, and the fan 13 is mounted below the control circuit board 19 and is close to the air outlets 122.

In a preferred embodiment of the invention, the control circuit board 19 is controllable to select the aromatizing mode of the air purifier or the aromatizing effect of the air purifying unit 100 (for example, by adjusting the air discharge volume) through an application (app) 210 of a mobile device 200 by network transmission signals 220. The network transmission signals 220 may be Wi-Fi signals, Bluetooth signals, or Zigbee signals.

In a preferred embodiment of the invention, the filter 14 is made up of a filter material selected from the group consisting of a high efficiency particulate air (HEPA) filter, an ultra low penetration air (ULPA) filter, nano photocatalysts, an active carbon filter, and an electrostatic filter.

In a preferred embodiment of the invention, the top surface of the mounting seat 162 is provided with a round groove 163 for allowing the aromatizing piece 162 to be mounted therein. The bottom surface of the mounting seat 162 is provided with a multiplicity of legs 164 extending downwardly. The top surface of the bottom cover 17 is provided with a multiplicity of hollow cylinders 171 extending upwardly for receiving the legs 164 of the mounting seat 162 to secure the mounting seat 162.

In a preferred embodiment of the invention, a lucid ring body 125 is mounted between the upper housing 12 and the lower housing 11. Also, a light-emitting diode (LED) 126 is electrically connected to the control circuit board 19 and is disposed on the inside of the lucid ring body 125. The LED 126 is used as an indication of the power of the air purifying unit 100. When the switch 123 is depressed to turn on the air purifying unit 100, the LED 126 will illuminate such that light is emitted through the lucid ring body 125.

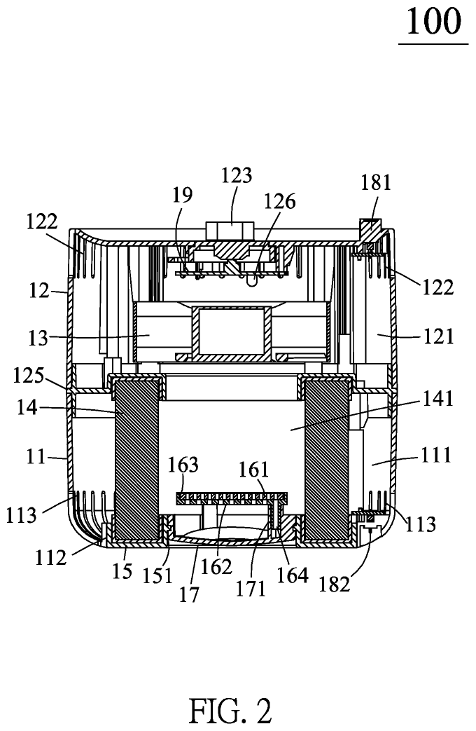

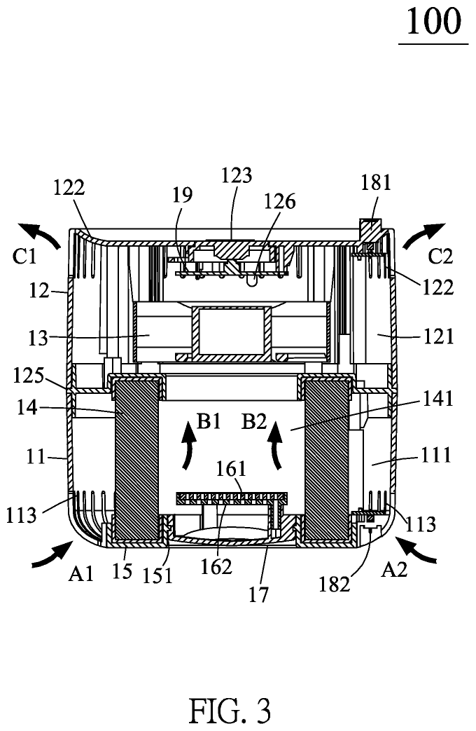

The operation of the air purifying unit 100 is described with reference to FIGS. 2 and 3, in which FIG. 2 is a cross-sectional view of the air purifier with the switch 123 not being depressed and FIG. 3 is a cross-sectional view of the air purifier with the switch 123 being depressed. As shown in FIG. 3, the switch 123 is depressed and thus the LED 126 illuminates, such that the light is emitted through the lucid ring body 125. In the meantime, the fan 23 starts to operate. The air enters the air purifying unit 100 through the air inlets 113, as indicated by the arrow-headed lines A1 and A2. The air is filtered by the filter 14 into purified air, which would pass through the aromatizing device 16 disposed in the hollow space 141 of the filter 14, such that the purified air is aromatized by the aromatizing piece 161 to generate purified and aromatized air. The fan 13 is used to blow the purified and aromatized air toward the upper housing 12, as indicated by the arrow-headed lines B1 and B2. Afterwards, the purified and aromatized air is discharged to the outside through the air outlets 122 of the air purifying unit 100, as indicated by the arrow-headed lines C1 and C2. In this way, the air purifying unit 100 can aromatize the air while purifying the air to generate purified and aromatized air, in order to disinfect, sterilize, and aromatize the indoor air of the place where the air purifying unit 100 is located. In brief, the invention can provide an air purifier with high portability and easy-to-operation characteristics. Thus, the invention possesses potentiality to contain the pandemic of virus.

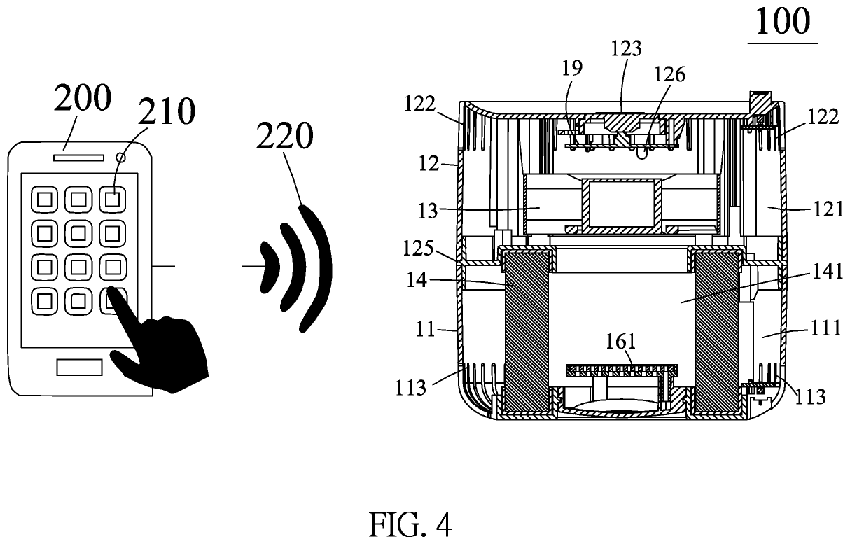

Please refer to FIGS. 3 and 4, in which FIG. 4 is a schematic diagram showing the air purifier combined with an aromatizing device being controllable by a mobile device. As shown, the air purifying unit 100 can be used solely. As the air purifying unit 100 includes a filter 14 and an aromatizing device 16, a single purifying unit 100 is adequate for the purification and aromatization of the fir. Also, as the air purifying unit 100 includes a control circuit board 19, the user may use a mobile device 200 to execute an application (APP) 210 installed in the mobile device 200 to control the operation of the air purifying unit 100 through the selection of different aromatizing mode for the air purifier by network transmission signals 220.

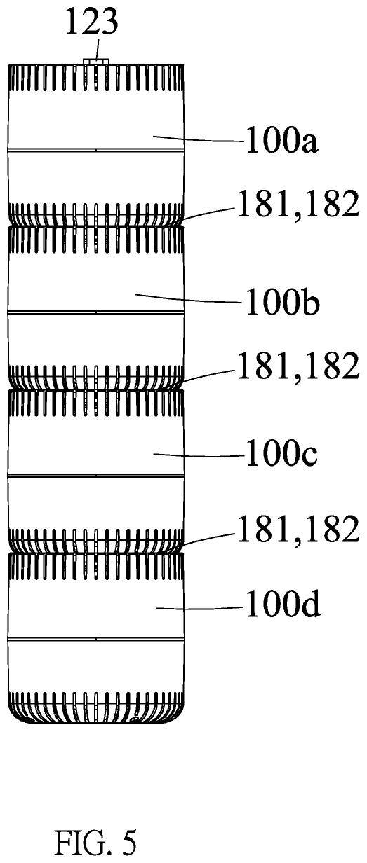

Please refer to FIGS. 3-5, in which FIG. 5 is a schematic diagram showing the air purifier being implemented by cascading a multiplicity of air purifying units. As shown, the air purifying unit 100 further includes a first extension connector 181 mounted on the top surface of the upper housing 12, and a second extension connector 182 mounted on mounted on the bottom surface of the lower housing 11. Hence, the inventive air purifier can be implemented by a single air purifying unit 100 as shown in FIG. 3 or implemented by a multiplicity of air purifying units 100a-100d that are vertically cascaded as shown in FIG. 5. In FIG. 5, each of the air purifying unit 100a-100d includes a first extension connector 181 mounted on the top surface of the upper housing and a second extension connector 182 mounted on mounted on the bottom surface of the lower housing, such that all of the air purifying unit 100a-100d can be electrically interconnected when they are cascaded. The user may use the application 210 of the mobile device 200 to select a desired aromatizing mode by sending the related messages to the air purifying unit 100a-100d by network transmission signals 220.

In this embodiment, each aromatizing device 16 of the air purifiers 100a-100d may be implemented by a specific kind of aromatizing piece 161. For example, the aromatizing piece 161 may be a bergamot oil aromatizing piece, a geranium oil aromatizing piece, a cedarwood oil aromatizing piece, or a lavender oil aromatizing piece. Therefore, the user may select the aromatizing mode through the application 210 of the mobile device 200. For example, when one of the air purifying units is selected, the selected air purifying unit can generate purified air with a single aroma. When two or more air purifying units are selected, the selected air purifying units can generate purified air with a mixed aroma. In this embodiment, the number of the air purifying units is set to four. However, the number of the air purifying units may be set to any arbitrary number.

Although the structure and the operation of the inventive air purifier combined with aromatizing device have been described herein, it is to be noted that those of skill in the art will recognize that various modifications can be made within the spirit and scope of the present invention as further defined in the appended claims.

What is claimed is:

1. An air purifier combined with an aromatizing device, characterized in that the air purifier comprises at least one air purifying unit having a lower housing, an upper housing, a fan, a filter, a filter cover, an aromatizing device, and a bottom cover, wherein:
the lower housing is a hollow housing having a lower accommodating space provided with a lower opening for receiving the filter and the aromatizing device, in which a bottom surface of the lower housing is provided with air outlets at circumferences thereof for allowing external air to enter the air purifying unit;
the upper housing is a hollow housing having an upper accommodating space and provided with a switch for turning on or off operations of the air purifying unit, in which a top surface of the upper housing is provided with air inlets at circumferences thereof for discharging purified and aromatized air;
the fan is mounted within the upper accommodating space of the upper housing for blowing the purified and aromatized air to the air inlets;
the filter is a hollow and cylindrical filter body having a hollow space for filtering the external air;
the filter cover is an annular body disposed between the lower side of the filter and the lower opening of the lower housing;
the aromatizing device comprises an aromatizing piece and a mounting seat, both of which are disposed within the hollow space of the filter for aromatizing purified air from the filter to generate purified and aromatized air; and
the bottom cover is used to seal a hollow portion of the filter cover;
wherein the air purifying unit further includes a first extension connector mounted on the top surface of the upper housing and a second extension connector mounted on the bottom surface of the lower housing, and wherein the first extension connector and the second extension connector are mated in a male-to-female configuration, such that the air purifying unit can be used solely, or jointly by vertically cascading a multiplicity of the air purifying units.

2. The air purifier combined with an aromatizing device according to claim 1 wherein the upper housing is further provided with a control circuit board electrically connected to the switch, the first extension connector, the second extension connector, and the fan and mounted on a bottom surface of the switch, and wherein the fan is mounted below the control circuit board and is close to the air outlets.

3. The air purifier combined with an aromatizing device according to claim 2 wherein the control circuit board is controllable to select an aromatizing mode of the air purifier through an application (app) of a mobile device by network transmission signals.

4. The air purifier combined with an aromatizing device according to claim 2 wherein the filter is made up of a filter material selected from the group consisting of a high efficiency particulate air (HEPA) filter, an ultra low penetration air (ULPA) filter, nano photocatalysts, an active carbon filter, and an electrostatic filter.

5. The air purifier combined with an aromatizing device according to claim 2 wherein the mounting seat is provided with a round groove a round groove for allowing the aromatizing piece to be mounted therein, and a bottom surface of the mounting seat is provided with a multiplicity of legs extending downwardly, and a top surface of the bottom cover is provided with a multiplicity of hollow cylinders extending upwardly for receiving legs of the mounting seat to secure the mounting seat.

6. The air purifier combined with an aromatizing device according to claim 2 further comprising a lucid ring body mounted between the upper housing and the lower housing, and a light-emitting diode connected to the control circuit board and disposed on an inside of the lucid ring body.
